(19) United States
(12) Patent Application Publication
(10) Pub. No.: US 2006/0154334 A1
(43) Pub. Date: Jul. 13, 2006

(54) Method of producing an antibody to epidermal growth factor receptor

(76) Inventors: Joseph Tarnowski, Stirling, NJ (US); Daniel Velez, Flemington, NJ (US); Joel Goldstein, Flemington, NJ (US); Michael Barry, Flemington, NJ (US); Diane Blumenthal, Yardley, PA (US); Girish Pendse, Hillsborough, NJ (US)

Correspondence Address: KENYON & KENYON LLP, ONE BROADWAY, NEW YORK, NY, 10004, US

(21) Appl. No.: 11/232,150

(22) Filed: Sep. 20, 2005

Related U.S. Application Data
(63) Continuation-in-part of application No. PCT/US04/08802, filed on Mar. 22, 2004.
(60) Provisional application No. 60/456,324, filed on Mar. 20, 2003.
(60) Provisional application No. 60/523,836, filed on Nov. 19, 2003.

(51) Int. Cl. C07K 16/28; C07H 21/04; C12P 21/06; C12N 5/06

(52) U.S. Cl. 435/69.1; 435/334; 435/320.1; 530/388.22; 536/23.53

(57) ABSTRACT

The present invention is directed to a method of producing an antibody to Epidermal Growth Factor Receptor (EGFR). The method includes producing transformed cells that express EGFR antibodies, culturing the transformed cells, harvesting the transformed cells to collect the EGFR antibodies, and purifying the EGFR antibodies.

### RELATED APPLICATIONS

This application is a continuation-in-part and claims the benefit under 35 U.S.C. § 120 of PCT International Application No. PCT/US2004/008802, filed on Mar. 22, 2004 and claims the benefit under 35 U.S.C. §119 of U.S. Provisional Application Nos. 60/456,324, filed on Mar. 20, 2003 and 60/523,836, filed on Nov. 19, 2003.

### FIELD OF THE INVENTION

The present invention relates to a method of producing an antibody specific for Epidermal Growth Factor Receptor.

### BACKGROUND OF THE INVENTION

Angiogenesis, which refers to the formation of capillaries from pre-existing vessels in the embryo and adult organism, is known to be a key element in tumor growth, survival and metastasis. Growth factors and their receptors, including epidermal growth factor (EGF) and transforming growth factor-α (TGF-α), which activate EGFR, are thought to play a role in tumor angiogenesis. Binding of these growth factors to their cell surface receptors induces receptor activation, which initiates and modifies signal transduction pathways and leads to cell proliferation and differentiation.

EGFR is a 170 kD membrane-spanning glycoprotein with an extracellular ligand binding domain, a transmembrane region and a cytoplasmic protein tyrosine kinase domain. See, e.g., Baselga et al., Epidermal Growth Factor Receptor: Potential Target for Anti-tumor Agents, The Center for Biomedical Continuing Education (2000). Binding of specific ligands, such as EGF and TNF-α, to EFGR results in EGFR autophosphorylation, activation of the receptor's cytoplasmic tyrosine kinase domain, and initiation of multiple signal transduction pathways that regulate tumor growth and survival. The EGFR pathway also influences production of various other angiogenic factors, such as VEGF and basis fibroblastic growth factor (bFGF), in tumors.

Previous studies directed to blocking EGFR have demonstrated that such a blockade can inhibit tumor growth. Various different inhibitors of EGFR have been utilized; for example, EGFR-specific small molecules and monoclonal antibodies have been developed, including the monoclonal antibody cetuximab, which is currently in clinical trials.

Current methods of producing EGFR antibodies, however, have not resulted in a significant yield of the antibodies. Accordingly, there is an unmet need in the art for a method of producing a high yield of EGFR antibodies.

### SUMMARY OF THE INVENTION

The present invention is directed to a method of producing antibodies to EGFR. The method includes producing transformed cells that express the EGFR antibodies, culturing the transformed cells, harvesting the transformed cells to collect the EGFR antibodies, and purifying the EGFR antibodies. In particular, the method involves selecting a transformant with DNA that encodes an EGFR antibody, cultivating the transformant in an inoculum cultivation medium to create an inoculum, scaling-up the inoculum in scale-up medium, stirring the inoculum in a production medium to produce and accumulate EGFR antibodies in a culture, harvesting the EGFR antibodies from the culture, and purifying the EGFR antibodies.

### BRIEF DESCRIPTION OF THE DRAWINGS

FIG. 1 is the heavy chain cDNA sequence (SEQ ID NO: 1) and the encoded amino acid sequence (SEQ ID NO: 2).

FIG. 2 is the light chain cDNA sequence (SEQ ID NO: 3) and the encoded amino acid sequence (SEQ ID NO: 4).

FIG. 3 is the amino acid sequence (SEQ ID NO: 2) of the heavy chain with the signal sequence italicized, the CDRs underlined, and the constant region bolded. The beginning of the constant region is indicated by (−).

FIG. 4 is the amino acid sequence (SEQ ID NO: 4) of the light chain with the signal sequence italicized, the CDRs underlined, and the constant region bolded. The beginning of the constant region is indicated by (−).

### DETAILED DESCRIPTION OF THE INVENTION

The present invention relates to a method of producing antibodies to EGFR. The antibodies of the present invention can be monoclonal or polyclonal antibodies or any other suitable type of an antibody, such as a fragment or a derivative of an antibody, a single chain antibody (scFv) or a synthetic homologue of the antibody, provided that the antibody has the same binding characteristics as, or that have binding characteristics comparable to, those of the whole antibody. As used herein, unless otherwise indicated or clear from the context, antibody domains, regions and fragments are accorded standard definitions as are well known in the art. See, e.g., Abbas et al., Cellular and Molecular Immunology, W.B. Saunders Company, Philadelphia, Pa. (1991).

Cleaving a whole antibody can produce antibody fragments, or by expressing DNA that encodes the fragment. Fragments of antibodies can be prepared by methods described by Lamoyi et al., J. Immunol. Methods, 56: 235-243 (1983) and by Parham, J. Immunol. 131: 2895-2902 (1983). Such fragments can contain one or both Fab fragments or the F(ab′)₂ fragment. Such fragments can also contain single-chain fragment variable region antibodies, i.e. scFv, dibodies, or other antibody fragments. Preferably the antibody fragments contain all six complementarity-determining regions of the whole antibody, although fragments containing fewer than all of such regions, such as three, four or five CDRs, can also be functional. The antibody fragment can also be conjugated to a carrier molecule. Some suitable carrier molecules include keyhole limpet hemocyanin and bovine serum albumen. Conjugation can be carried out by methods known in the art.

Antibodies of the present invention also include those for which binding characteristics have been improved by direct mutation, methods of affinity maturation, phage display, or chain shuffling. Affinity and specificity can be modified or improved by mutating CDRs and screening for antigen binding sites having the desired characteristics (see, e.g., Yang et al., J. Mol. Bio., 254: 392-403 (1995)). CDRs are mutated in a variety of ways. One way is to randomize individual residues or combinations of residues so that in a population of otherwise identical antigen binding sites, all twenty amino acids are found at particular positions. Alternatively, mutations are induced over a range of CDR residues by error prone PCR methods (see, e.g., Hawkins et al., J. Mol. Bio., 226: 889-896 (1992)). Phage display vectors containing heavy and light chain variable region genes are propagated in mutator strains of E. coli (see, e.g., Low et al., J. Mol. Bio., 250: 359-368 (1996)). These methods of mutagenesis are illustrative of the many methods known to one of skill in the art.

The antibodies of the present invention can also be bispecific and/or multivalent. A variety of chemical and recombinant methods have been developed for the production of bispecific and/or multivalent antibody fragments. For a review, see Holliger and Winter, Curr. Opin. Biotechnol. 4: 446-449 (1993); Carter et al., J. Hematotherapy 4:463-470 (1995); Plückthun and Pack, Immunotechnology 3, 83-105 (1997). Bispecificity and/or bivalency has been accomplished by fusing two scFv molecules via flexible linkers, leucine zipper motifs, C_{H}C_{L}-heterodimerization, and by association of scFv molecules to form bivalent monospecific diabodies and related structures. The addition of multimerization sequences at the carboxy or amino terminus of the scFv or Fab fragments has achieved multivalency, by using, for example, p53, streptavidin, and helix-turn-helix motifs. For example, by dimerization via the helix-turn-helix motif of an scFv fusion protein of the form (scFv1)-hinge-helix-tum-helix-(scFv2), a tetravalent bispecific miniantibody is produced having two scFv binding sites for each of two target antigens. Improved avidity can also been obtained by providing three functional antigen binding sites. For example, scFv molecules with shortened linkers connecting the V_{H} and V_{L} domains associate to form a triabody (Kortt et al., Protein Eng. 10:423-433 (1997)).

Production of IgG-type bispecific antibodies, which resemble IgG antibodies in that they possess a more or less complete IgG constant domain structure, has been achieved by chemical cross-linking of two different IgG molecules or by co-expression of two antibodies from the same cell. One strategy developed to overcome unwanted pairings between two different sets of IgG heavy and light chains co-expressed in transfected cells is modification of the C_{H}3 domains of two heavy chains to reduce homodimerization between like antibody heavy chains. Merchant et al., Nat. Biotechnology 16: 677-681 (1998). In that method, light chain mispairing was eliminated by requiring the use of identical light chains for each binding site of those bispecific antibodies.

In some cases, it is desirable to maintain functional or structural aspects other than antigen specificity. For example, both complement-mediated cytotoxicity (CMC) and antibody-dependent cell-mediated cytotoxicity (ADCC), which require the presence and function of Fc region heavy chain constant domains, are lost in most bispecific antibodies. Coloma and Morrison created a homogeneous population of bivalent BsAb molecules with an Fc domain by fusing a scFv to the C-terminus of a complete heavy chain. Co-expression of the fusion with an antibody light chain resulted in the production of a homogeneous population of bivalent, bispecific molecules that bind to one antigen at one end and to a second antigen at the other end (Coloma and Morrison, Nat. Biotechnology 15, 159-163 (1997)). However, this molecule had a reduced ability to activate complement and was incapable of effecting CMC. Furthermore, the C_{H}3 domain bound to high affinity Fc receptor (Fc(R1) with reduced affinity. Zhu et al., PCT/US01/16924, have described the replacement of Ig variable domains with single chain Fvs in order to produce tetrameric Ig-like proteins that (1) are bispecific and bivalent, (2) are substantially homogeneous with no constraints regarding selection of antigen-binding sites, (3) comprise Fc constant domains and retain associated functions, and (4) can be produced in mammalian or other cells without further processing. By a similar method, bispecific monovalent Fab-like proteins can be produced.

Preferably, the antibodies of the subject invention are monoclonal antibodies. The antibodies of the present invention are also preferably chimeric antibodies having a variable region of an antibody of one species, for example, a mouse, and a constant region of an antibody of a different species, for example, a human. Alternatively, the antibodies of the present invention can be humanized antibodies having hypervariable or complementarity-determining regions (CDRs) of an antibody from one species, for example, a mouse, and framework variable regions and a constant region of a human antibody. Also alternatively, the antibodies of the present invention can be human antibodies having both a constant region and a variable region of a human antibody.

In one embodiment of the present invention, the EGFR antibody is a fully human, monoclonal antibody specific for EGFR, such as, for example, ABX-EGF (Abgenix, Inc). ABX-EFG binds EGFR with high specificity, blocking binding of EGFR to both its ligands, EGF and TNF-alpha. The sequence and characterization of ABX-EGF is disclosed in U.S. Pat. No. 6,235,883 at col. 28, line 62 through col. 29, line 36 and in FIG. 29-34, which is incorporated by reference herein. See also Yang et al., Critical Rev. Oncol./Hematol., 38 (1): 7-23, 2001, which is also incorporated by reference herein.

In a preferred embodiment, the EGFR antibody is a humanized monoclonal antibody specific for EGFR with complementarity determining regions as disclosed in U.S. Pat. No. 4,943,533 to Mendelsohn et al (ATCC HB8506, HB8507, HB8508 and HB8509), which is incorporated by reference herein.

In a more preferred embodiment, the EGFR antibody is a chimeric antibody, such as, for example, cetuximab, which specifically binds EGFR and blocks binding of a ligand, such as EGF or TNF-α, to EGFR. This blockage results in inhibition of tumor growth, which includes inhibition of tumor invasion, metastasis, cell repair, and angiogenesis, by interfering with the effects of EGFR activation. In addition, or alternatively, cetuximab may promote internalization of the receptor-antibody complex, preventing further stimulation of the receptor by its ligand or any other mechanism. Further characterization of cetuximab is disclosed in U.S. application Ser. Nos. 08/973,065 to Goldstein et al., and 09/635,974 to Teufel; WO 99/60023 to Waksal et al., and WO 00/69459 to Waksal, all of which are incorporated by reference herein.

Notwithstanding the exact nature or characteristics of an EGFR antibody, the method of producing an EGFR antibody according to the present invention generally includes the steps of producing transformed cells that express EGFR antibodies (the transforming step), culturing the transformed cells (the culturing step), harvesting the transformed cells to collect the EGFR antibodies (the harvesting step), and purifying the EGFR antibodies (the purifying step).

With respect to the transforming step, a DNA encoding an EGFR antibody is isolated and inserted into a replicable vector for further cloning or for expression. The DNA encoding the EGFR antibody can be generated by methods known in the art, including, but are not limited to, production in hybridoma cells. These methods are described in various publications, including the immunological method described by Kohler and Milstein, Nature 256: 495-497 (1975) and Campbell in “Monoclonal Antibody Technology, The Production and Characterization of Rodent and Human Hybridomas” in Burdon et al., Eds., Laboratory Techniques in Biochemistry and Molecular Biology, Volume 13, Elsevier Science Publishers, Amsterdam (1985); as well as by the recombinant DNA methods described by Huse et al. in Science, 246: 1275-1281 (1989).

Methods for incorporating the DNA into a vector are well known in the art and include direct cloning, site specific recombination using recombinases, homologous recombination, and other suitable methods of constructing a recombinant vector. See generally, Sandbrook et al. Molecular Cloning: A Laboratory Manual 3^{rd} edition, Cold Spring Harbor Press (1989).

Vectors useful in the present invention are also well known in the art and include for example, bacterial or viral vectors. Suitable bacterial vectors include plasmids such as pBR322-based plasmids, Bluescript, pSKF, and pET23D, and bacteriophages, e.g., lambda and M13 based vectors. Suitable viral vectors include retroviral vectors, adenoviral vectors, adeno-associated viral vectors, herpesviral vectors, SV40 viral vectors, polyoma virus vectors, papilloma virus vectors, picnovirus vectors, vaccinia virus vectors, or other suitable vectors. DNA expression by a suitable vector can be controlled by inducible or uninducible regulatory sequences. Generally, a vector useful in the present invention can, therefore, also include any or all of the following: signal peptide, a leader sequence, one or more marker genes, a promoter, and a transcription termination sequence.

Once a suitable expression vector according to the present invention is identified, the expression vector is introduced into a host cell. Any suitable method of introducing the expression vector into a host cell can be employed, including calcium phosphate precipitation, nuclear injection, and electroporation, for example. The host cells of the present invention can include prokaryotic and eukaryotic organisms, such as, for example, mammalian cells. Preferably, the host cells are mammalian cells such as, for example, SP2/0 cells, NS0 cells, COS-7 cells, Chinese hamster ovary (CHO) cells, and cells lines of lymphoid origin, such as lymphoma, myeloma, or hybridoma cells, for example. Other eukaryotic host, such as yeasts and plants, can alternatively be used.

For those EGFR antibodies of the present invention that contain both a light and heavy chain, these chains can be transformed into separate cell cultures, either of the same or of differing species. Alternatively, the light and heavy chains can be co-transformed into a single cell culture by using separate vectors or a single expression vector that contains the coding genes for both the light and heavy chain.

During the culturing step, the transformed cells are cultured by preparing and cultivating an inoculum (the cultivation phase), scaling up the inoculum in a series of bioreactors (the scale-up phase), and producing and accumulating EGFR antibodies from the inoculum (the production phase).

In the cultivation phase, the transformed cells from the transforming step are recovered into an inoculum cultivation medium to create an inoculum. The transformed host cells are cultured by methods known in the art in a liquid medium containing assimilable sources of carbon (carbohydrates such as glucose or lactose), nitrogen (amino acids, peptides, proteins or their degradation products such as peptones, ammonium salts or the like), and inorganic salts (sulfates, phosphates and/or carbonates of sodium, potassium, magnesium and calcium). The inoculum cultivation medium preferably includes a conventional nutrient medium such as Dulbecco's Modified Eagle's Medium (DMEM) (Sigma), Ham's F10 (Sigma), Minimal Essential Medium (MEM) (Sigma), RPMI-1640 (Sigma) or NCTC-135.

Any of these media can be supplemented as necessary with amino acids (glutamine), hormones or other growth factors (insulin, transferrin, or epidermal growth factor), vitamins, salts (zinc sulfate, sodium chloride, phosphate), buffers, nucleotides, antibiotics, ionic surfactants, and glucose or an equivalent energy source. The medium can further contain trace elements that are growth promoting substances, such as iron chelates (e.g., chelate B, Invitrogen Corp., Carlsbad, Calif.), and manganese. During the cultivation phases, culture conditions, such as temperature, pH, and the like, are monitored to ensure rapid cell growth.

During the scale-up phase, the inoculum is scaled-up in scale-up medium through sequential steps of cultivation. Such steps can be performed in any suitable container, including cell culture flasks, stir bottles, roller bottles, rotary bioreactors, and spinner flasks. The scale-up medium also includes a conventional nutrient medium and can include amino acids supplied by hydrolysates (e.g., HySoy, Quest International, Chicago, Ill.), hormones or other growth factors, vitamins, salts, buffers, nucleotides, antibiotics, ionic surfactants, iron chelates, and glucose or an equivalent energy source. During the scale-up phase in bioreactors, the pH, oxygen saturation, and waste products of the inoculum are monitored.

During the production phase, the cells are transferred to a stir tank or airlift bioreactor and fed with a complex growth medium containing sugars, amino acids, salts, trace elements and growth factors, which are combined in such quantities so as to maintain the pH, osmolality, and other essential parameters of the growth medium for consistent, robust, rapid cell growth. The use of osmoprotectant compounds, such as betaine or proline, for example, can be used to protect cells from osmotic stress while enhancing antibody productivity. The temperature, dissolved oxygen, pH, pressure, gas flow rate, and stir rate are also controlled during the production phase. During the production phase, the cells develop within themselves the EGFR antibodies or secrete the EGFR antibodies into the surrounding medium as a by-product of growth. Those cells that develop EGFR antibodies within their structures can be chemically or mechanically fragmented in order to harvest the EFGR antibodies. More complex cells such as mammalian cells can produce sugar-modified cellular products and secrete the EGFR antibodies into the cell culture medium for isolation.

In another embodiment of the invention, the culturing step includes culturing and expanding the transformed cells in such a way that methotrexate (MTX) is present at all steps of the culturing process. Preferably, one cultivation media containing MTX is used to prepare and cultivate an inoculum of the transformed cells and to expand the inoculum in a series of bioreactors according to the methods of cultivation and scale-up described above.

The cultivation media used in this embodiment of the invention comprises a medium base powder containing salts (sulfates, phosphates and/or carbonates of sodium, potassium, magnesium and calcium), nitrogen (amino acids, peptides, proteins or their degradation products such as peptones and ammonium salts), assimilable sources of carbon (carbohydrates such as glucose or lactose), vitamins, and trace elements. The medium base power preferably includes a conventional nutrient medium such as Dulbecco's Modified Eagle's Medium (DMEM). The cultivation media also includes Hy-Soy® and EX-CYTE® supplements, a buffering agent (such as sodium bicarbonate), and MTX. In addition, the cultivation media may contain recombinant human insulin, bovine serum albumin, an anti-shearing agent (such as Pluronic F68), and Iron Chelate B, an aqueous solution of food grade ethyl maltol used for chelation of inorganic iron during cell culture. Preferably, the cultivation media comprises the ingredients in Table 7.

In this embodiment of the invention, the cultured and expanded cells are transferred to a stir tank or airlift bioreactor containing a production media with MTX, and enter the production phase according to the method previously described. The production media used in this embodiment of the invention comprises a medium base powder containing salts (sulfates, phosphates and/or carbonates of sodium, potassium, magnesium and calcium), nitrogen (amino acids, peptides, proteins or their degradation products such as peptones and ammonium salts), assimilable sources of carbon (carbohydrates such as glucose or lactose), vitamins, and trace elements. The medium base power preferably includes a conventional nutrient medium such as Dulbecco's Modified Eagle's Medium (DMEM). The production media also includes Hy-SOY® and EX-CYTE® supplements, a buffering agent (such as sodium bicarbonate), MTX, and hydrocortisone. Preferably, the production media comprises the ingredients in Table 8.

In one embodiment of the invention, EX-CYTE®, glucose, glutamine, and Hy-Soy® nutrients are added during the course of the cell culture production process to support the higher cell densities achieved during this process phase. Cell density criteria are used to determine when the culture is fed additional nutrients. The acceptable viable cell concentration prior to initiation of the first nutrient feed, which preferably comprises EX-CYTE® and a glutamine/glucose solution) is ≧6.0×10⁵ cells/mL. The second feed, which preferably comprises Hy-Soy®, occurs when a viable cell concentration of ≧2.0×10⁶ cells/mL is achieved, or one day after peak viable cell density. The pH set point of the culture is preferably lowered to pH 6.9 about 16 to 30 hours after the first nutrient addition in order to facilitate production.

During the harvesting step, the EGFR antibodies are removed from the cell culture by any means known in the art. For example, when the EGFR antibodies are produced intracellularly by the transformed cells, centrifugation or ultrafiltration can be used to remove the host cells or lysed cells. Where the EGFR antibodies are secreted into the medium, the antibodies can be removed from the mixture of compounds fed to the cells and from the by-products of the cells themselves by using commercially available protein concentration filters, such as, for example, Amicon or Millipore Pellicon ultrafiltration units.

During the purifying step, the EGFR antibodies are subjected to one or more purification steps, including various chromatography methods. Examples of such purification procedures include anion exchange chromatography and cation exchange chromatography, as well as various filtration methods, such as tangential flow filtration using Pellicon™ membranes (Millipore, Billerica, Mass.), for example, nanofiltration using DVSO filters (Pall Corporation, East Hills, N.Y.), for example, reduce potential viral contamination and appropriate size dead end filtration (such as 0.45 μm and 0.2 μm filters), fractionation on a hydrophobic interaction chromatography (e.g. on phenyl sepharose), ethanol precipitation, isoelectric focusing, Reverse Phase HPLC, chromatography on silica, chromatography on HEPARIN SEPHAROSE,™ further ion exchange chromatography, chromatofocusing, SDS-PAGE, ammonium sulfate precipitation, hydroxylapatite chromatography, gel electrophoresis, dialysis, and affinity chromatography (e.g., using protein A, protein G, an antibody, a specific substrate, ligand or antigen as the capture reagent).

The antibodies of the present invention can also be modified or derivatized. Examples of such modification include post-translation modifications, such as glycosylation (both O-linked and N-linked), acetylation, phosphorylation, ubiquitination, and the like. These modifications can be carried out in vivo using the host cell machinery or in vitro following isolation of the antibody from the host cell.

It is understood that the EGFR antibodies of the invention can be mixed with a pharmaceutically acceptable carrier, or diluted by a carrier, and/or enclosed within a carrier, which can, for example, be in the form of a capsule, sachet, paper or other container. When the carrier serves as a diluent, it can be a solid, semi-solid, or liquid material, which acts as a vehicle, excipient or medium for the active ingredient. Suitable pharmaceutically acceptable carriers include, for example, one or more of water, saline, phosphate buffered saline, dextrose, glycerol, ethanol and the like, as well as combinations thereof. Pharmaceutically acceptable carriers can further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient.

The EGFR antibodies of this invention can also be in a variety of forms. These include, for example, solid, semi-solid and liquid dosage forms, such as tablets, pills, powders, liquid solutions, dispersions or suspensions, liposomes, suppositories, injectable and infusible solutions. Thus, the composition be in the form of tablets, lozenges, sachets, cachets, elixirs, suspensions, aerosols (as a solid or in a liquid medium), ointments containing for example up to 10% by weight of the active compound, soft and hard gelatin capsules, suppositories, injection solutions, suspensions, sterile packaged powders and as a topical patch. The preferred form depends on the intended mode of administration and therapeutic application.

### EXAMPLES

### Example 1

Producing Transformed Cells that Express EGFR Antibodies

The myeloma cell line SP2/0-Ag14 (ATCC CRL-1581), which is a line that was formed by fusing BALB/c spleen cells (from mouse immunized with sheep RBCs) with the P3X63Ag8 myeloma (see Shulman et al., Nature 276: 269-270 (1978)) was transformed to express EGFR antibodies. The cell line was expanded in tissue culture flasks (1L) and total cell RNA was prepared by lysing washed cells in gaunidine isothiocyanate containing 2-mercaptoethanol (25 mL), shearing the solution in a dounce homogenizer to degrade cell DNA, and layering the preparation on a CsCl cushion (10 mL). After centrifugation (24,000 rpm, 16 hrs), the RNA pellet was resuspended in TE buffer and precipitated with ethanol. The poly A (+) mRNA fraction was isolated by binding to and elution from oligo dT cellulose.

A cDNA library was prepared using the poly A(+) mRNA as template and oligo dt as primer. The second strand was synthesized by nick translation using RNase H and DNA polymerase I. The double stranded DNA was passed through a G-75 Sepharose column (2 mL) to remove oligo dT and small products and then ligated to a polylinker with the sequence: 5′-AATTCTCGAGTCTAGA-3′ (SEQ ID NO: 5) encoding an EcoRI four base sticky end for ligation to the cloning vector, and the restriction sites for XhoI and XbaI for subsequent manipulations of the cDNAs. The ligated cDNA was then size-selected to enrich for full length by electrophoresis on a 5% polyacrylamide gel. The appropriate size fractions (˜1500 bp for H chain and ˜900 bp for L chain cDNA) were electroeluted from gel slices and ligated to EcoRI-digested lambda gt10 phage DNA.

Libraries were generated by packaging the ligation products in vitro and plating the recombinant phage on lawns of E. coli strain C600 HFL. Phage containing H and L cDNAs were identified by phage filter lifts that were hybridized with radiolabeled oligonucleotides specific for the mouse kappa and gamma constant regions.

Plaque purified, hybridization-positive phage were analyzed by restriction digestion and agarose gel electrophoresis. Isolates with the longest cDNA inserts were subcloned in a plasmid vector and analyzed by DNA sequencing.

In order to identify the correct L chain cDNA, a sample of mouse EGFR antibody was sequenced by automated Edman degradation after first separating H and L chains by reducing SDS gel electrophoresis and blotting to membranes. The sequence obtained for the L chain matched one of the cDNAs.

The V regions were adapted for expression by ligating the body of each to a synthetic DNA duplex encoding the sequence between the closest unique restriction site to the V/C junction and the exact boundary of the V region. To this was ligated a second short intron sequence, which when joined restores a functional splice donor site to the V region. At the end of the intron for the L chain is a BaMHI site and at the end of the H chain intron is a HindIII site. The adapted L chain V region was then isolated as a XbaI-BamHI fragment (the XbaI site was in the original linker used for cDNA cloning) while the adapted H chain V region was isolated as a XhoI-HindIII fragment.

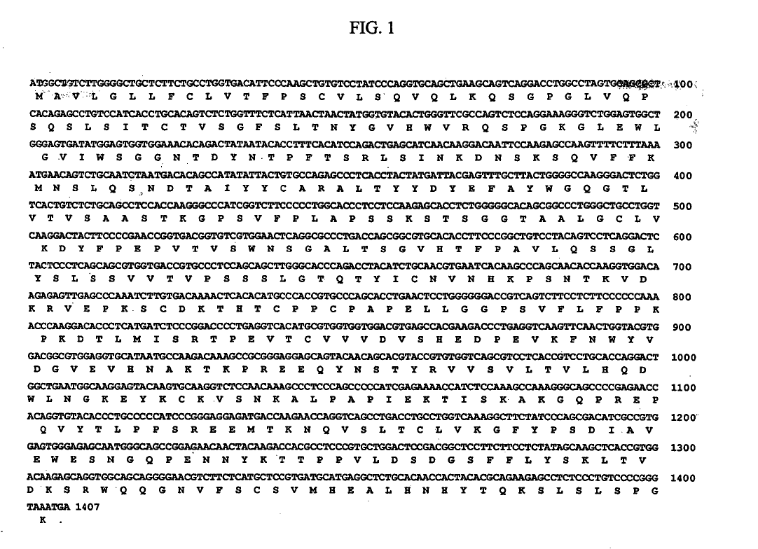

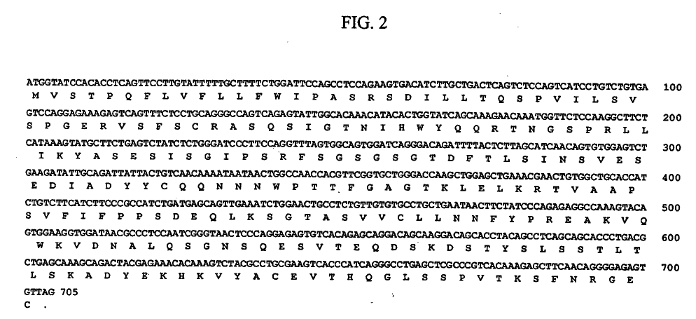

The expression vector, containing human kappa and human gamma 1 constant regions, was digested with XbaI and BamHI and used for the insertion of the adapted light chain variable region. The resulting plasmid was then digested with XhoI and HindIII and used for the insertion of the adapted H chain V region. The final vector for expression of the EGFR antibody was identified by restriction analyses. Set forth in FIG. 1 is the nucleotide sequence of the heavy chain cDNA and in FIG. 2 is the nucleotide sequence of the light chain cDNA.

The final vector was introduced into hybridoma sp2/0 Ag14 cells by protoplast fusion. The bacteria harboring the vector were grown to an optical density of 0.5 at 600 nm at which time chloramphenicol was added to arrest growth and amplify the vector copy number. The following day the bacteria were treated with lysozyme to remove the cell wall and the resulting protoplasts were fused to the hybridoma cells with polyethylene glycol (1500 mL). After the fusion, the cells were grown in antibiotics to kill any surviving bacteria and were plated in 96-well microtiter plates. The selection medium [containing methotrexate (MTX) at 0.1 μM] was added after 24-48 hr to allow only the transfected cells to grow, by virtue of their expression of the marker gene (dihydrofolate reductase) present in the expression plasmid.

After two weeks, several MTX-resistant clones were obtained that were then tested for antibody expression. Culture supernatants were added to wells coated with an anti-human Ig (Fc-specific) antibody as the capture reagent. The detection system was an HRP-conjugated goat anti-human kappa antibody. The majority of clones were found to be secreting human antibody determinants and the three highest producers were adapted to grow at 1 and then 5 μM MTX. The lines were subcloned by limiting dilution and the productivity of the subclones was tested by seeding cells at 2×10⁵ cells per mL in growth medium and measuring the accumulated antibody on day 7. The cell lines were subclones again and they all produced between 110 and 130 mg/L of antibody in the 7-day production assay.

### Example 2

Preparing and Cultivating Inoculum

Transformed cells from Example 1 were recovered into an inoculum cultivation medium that included the components listed in Table 1 (referred to herein as “Inoculum Cultivation Medium A.”)

TABLE 1

| Ingredient | Amount | |
|---|---|---|
| Dulbecco's Modified Eagle's Medium (DMEM) | 90% | |
| NCTC-135 | 10% | |
| Glutamine | 4 | mM |
| Bovine Insulin | 7.5 | mg/L |
| Bovine Transferrin | 7.5 | mg/L |
| Bovine Serum Albumen (BSA) | 1.0 | g/L |
| Ethanolamine | 30 | μM |
| Selenium | 40 | nM |
| Mercaptoethanol | 30 | μM |
| Oxaloacetate | 150 | mg/L |

### Example 3

Preparing and Cultivating an Inoculum

Transformed cells from Example 1 were recovered into an inoculum cultivation medium that included the components listed in Table 2 (referred to herein as “Inoculum Cultivation Medium B”). Inoculum Cultivation Medium B differed from Inoculum Cultivation Medium A in that bovine insulin was replaced with recombinant human insulin and bovine transferrin was replaced with an inorganic iron chelator. In addition, the concentration of amino acids, salts, and vitamins in DMEM and NCTC-135 and the concentration of glutamine were approximately doubled to that present in Inoculum Cultivation Medium A. Further, an inorganic salt, such as zinc sulfate, and an ionic surfactant, such as pluronic F68 were added to the inoculum cultivation medium.

TABLE 2

| Ingredient | Amount | |
|---|---|---|
| DMEM | 90% | |
| NCTC-135 | 10% | |
| Glutamine | 8 | mM |
| Human Recombinant Insulin | 20.0 | mg/L |
| Inorganic chelate (inorganic iron chelator) | 7.5 | mg/L |
| BSA | 1.0 | g/L |
| Ethanolamine | 30 | μM |
| Oxaloacetate | 150 | mg/L |
| Selenium | 40 | nM |
| Mercaptoethanol | 30 | μM |
| Zinc Sulfate | 1 | μM |
| Pluronic F68 | 1 | g/L |

### Example 4

Scaling-Up Inoculum

Inoculum created from Example 2 was scaled up in scale-up medium through sequential steps of cultivation in cell culture flasks, roller bottles, and spinner flasks. The scale-up medium included the components listed in Table 3 (referred to herein as “Scale-Up Medium A”).

TABLE 3

| Ingredient | Amount | |
|---|---|---|
| Inoculum Cultivation Medium A | 14.83 | g/L |
| Sodium Bicarbonate | 3.55 | g/L |
| Pluronic F68 | 1 | g/L |
| Methotrexate | 5 | μM |
| HySoy, UF (Quest); | 1.25 | g/L |
| Excyte VLE | 5.0 | mL/L |

### Example 5

Scaling-Up Inoculum

Inoculum from Example 3 was scaled up in scale-up medium through sequential steps of cultivation in cell culture flasks, roller bottles, and spinner flasks. The scale-up medium included the components listed in Table 4 (referred to herein as “Scale-Up Medium B”). Scale-Up Medium B differed from Scale-Up Medium A in that Inoculum Cultivation Medium B was used instead of Inoculum Cultivation Medium A. In addition, chelate B obtained from Invitrogen was added and pluronic F68 was eliminated.

TABLE 4

| Ingredient | Amount | |
|---|---|---|
| Inoculum Cultivation Medium B | 18.67 | g/L |
| Sodium Bicarbonate | 3.55 | g/L |
| Chelate B (Invitrogen) | 2.0 | mL/L |
| Methotrexate | 5 | μM |
| HySoy, UF (Quest); | 1.25 | g/L |
| Excyte VLE | 5.0 | mL/L |

### Example 6

Production and Accumulation of EGFR Antibodies

Inoculum from Example 4 was transferred to a 1,200 L stir tank. Production medium included the components listed in Table 5 (referred to herein as “Production Medium A”).

TABLE 5

| Ingredient | Amount | |
|---|---|---|
| Inoculum Cultivation Medium A | 14.83 | g/L |
| Sodium Bicarbonate | 3.55 | g/L |
| HySoy, UF (Quest); | 1.25 | g/L |
| Methotrexate | 5 | μM |
| Excyte VLE | 5.0 | mL/L |
| Hydrocortisone | 0.5 | μM |

Insulin, glutamine, and Excyte were added to the stir tank about three days (Day 3) after the inoculum was tranferred to the stir tank. Excyte was added after four days (Day 4) and then after five days (Day 5) after the inoculum was transferred. Glutamine control after two feeds (greater than 220 mg/L); glucose control after two feed (greater than 2.0 g/L). The pH of the contents of the stir tank was controlled at between 6.9 and 7.1 at Day 4, or 16-30 hours after the glucose, glutamine and ExCyte were added to the stir tank.

Temperature, dissolved oxygen, pH, pressure and gas flow rate during the production phase were controlled.

### Example 7

Production and Accumulation of EGFR Antibodies

The inoculum from Example 5 was transferred to a 12,000 L stir tank. Production differed from Example 6 in that Inoculum Cultivation Medium B was used instead of Inoculum Cultivation Medium A. In addition, chelate B was added. The production medium included the components listed in Table 6 (referred to herein as “Production Medium B”).

TABLE 6

| Ingredient | Amount | |
|---|---|---|
| Inoculum Cultivation Medium B | 18.67 | g/L |
| Sodium Bicarbonate | 3.55 | g/L |
| Inorganic Iron Chelate | 2.0 | mL/L |
| HySoy, UF (Quest) | 1.25 | g/L |
| Methotrexate | 5 | μM |
| Excyte VLE | 5.0 | mL/L |
| Hydrocortisone | 0.5 | μM |

Twenty millimoles of glucose, 20 mM of glutamine, and 15 mL/L of ExCyte were added to the stir tank approximately 48 hours after the inoculum was transferred to the stir tank. At this time, under preferred conditions, there were greater than 7.0×10⁵ viable cells/mL in the stir tank. Twenty-three point five grams per liter of HySoy was added at Day 4 or Day 5 or when the cell concentration was ≧2×10⁶ viable cells/mL. The pH of the contents of the stir tank was controlled at 6.9 at Day 4, or 16-30 hours after the glucose, glutamine and ExCyte were added to the stir tank.

Alternatively, 20 millimoles of glucose, 20 mM of glutamine, 15 mL/L of ExCyte and 90 mM Betaine were added to the stir tank approximately 48 hours after the inoculum was transferred to the stir tank. At this time, under preferred conditions, there were greater than 7.0×10⁵ viable cells/mL in the stir tank. Twenty-three point five grams per liter of HySoy was added at Day 4 or Day 5 or when the cell concentration was ≧2×10⁶ viable cells/mL. The pH of the contents of the stir tank was controlled at 6.9 at Day 4, or 16-30 hours after the glucose, glutamine and ExCyte were added to the stir tank.

Temperature, dissolved oxygen, pH, pressure and gas flow rate were controlled.

### Example 8

Cultivation of Inoculum, Scale-Up, and Production of EGFR Antibodies with MTX

Examples 8 and 9 describe a series of manufacturing steps in which methotrexate is used for innoculum preparation and cultivation, scaling-up and production. Transformed cells according to Example 1 were recovered from a frozen cell bank into a cultivation/scale-up medium that included the components listed in Table 7 (referred to herein as “innoculum/scale-up medium A”). The inoculum thus created was scaled up in the same medium through sequential steps of cultivation in cell culture flasks, roller bottles, and spinner flasks.

TABLE 7

| Ingredient | Amount |
|---|---|
| Dulbecco's Modified Eagle's Medium (DMEM) | 90%^{#} |
| NCTC-135 | 10%^{#} |
| Glutamine | 8 mM |
| Bovine Serum Albumen (BSA) | 1.0 g/L |
| Ethanolamine | 30 μM |
| Selenium | 40 μM |
| Mercaptoethanol | 30 μM |
| Oxaloacetate | 150 mg/L |
| Zinc Sulfate | 1 μM |
| Pluronic F68 | 1 g/L |
| Sodium Bicarbonate | 3.55 g/L |
| Iron Chelate B(ethyl maltol) | 100 μM |
| Methotrexate | 5 μM |
| HySoy, UF (Quest) | 1.25 g/L |
| EX-CYTE VLE | 5.0 mL/L |
| 6N Hydrochloric acid solution | as needed to adjust pH |
| 10 N Sodium hydroxide solution | as needed to adjust pH |

*DMEM and NCTG-135 included ˜2x concentration of certain amino acids, salts and vitamins.

The scaled-up innoculum was transferred to a 12,000 L stir tank containing production medium including the components listed in Table 8. As noted above in Example 3, the concentration of various amino acids, salts, and vitamins in DMEM and NCTC-135 were increased. For example, relative to standard DMEM formulations, amino acids were present at about 2× to 4×, ferric nitrate, magnesium sulfate, potassium chloride and sodium phosphate salts were present at about 2×, and folic acid was present at about 2×.

TABLE 8

| Ingredient | Amount |
|---|---|
| DMEM | 90%* |
| NCTC-135 | 10%* |
| Glutamine | 8 mM |
| Recombinant Human Insulin | 20 mg/L |
| BSA | 1.0 g/L |
| Ethanolamine | 30 μM |
| Selenium | 40 μM |
| Mercaptoethanol | 30 μM |
| Oxaloacetate | 150 mg/L |
| Zinc Sulfate | 1 μM |
| Pluronic F68 | 1 g/L |
| Sodium Bicarbonate | 3.55 g/L |
| Iron Chelate B (ethyl maltol) | 100 μM |
| Methotrexate | 5 μM |
| HySoy, UF (Quest) | 1.25 g/L |
| EX-CYTE VLE | 5.0 mL/L |
| 6N Hydrochloric acid solution | as needed to adjust pH |
| 10 N Sodium hydroxide solution | as needed to adjust pH |
| Hydrocortisone | 0.5 μM |

*DMEM and NCTG-135 included ˜2x concentration of certain amino acids, salts and vitamins.

Cell density criteria were monitored to determine when to supplement the media with additional nutrients. Upon reaching a viable cell concentration of ≧6.0×10⁵ cells/mL, EX-CYTE® and a glutamine/glucose solution were added (the first nutrient feed). A second feed, comprising Hy-Soy, was added when a viable cell concentration of ≧2.0×10⁶ cells/mL was achieved, or one day after peak viable cell density occured. The pH set point of the culture is preferably lowered to pH 6.9 about 16 to 30 hours after the first nutrient addition in order to facilitate production. Temperature, dissolved oxygen, pH, pressure and gas flow rate during the production phase were controlled.

### Example 9

Cultivation of Inoculum, Scale-Up, and Production of EGFR Antibodies with MTX

Transformed cells according to Example 1 were recovered from a frozen cell bank into a cultivation/scale-up medium that included the components listed in Table 3 (Example 4). The inoculum thus created was scaled up in the same medium through sequential steps of cultivation in cell culture flasks, roller bottles, and spinner flasks. The scaled-up innoculum was transferred to a stir tank containing production medium including the components listed in Table 5 (Example 6). Cell density criteria were monitored to determine when to supplement the media with additional nutrients. At Day 3 following transfer to the stir tank, EX-CYTE®, bovine insulin, and a glutamine/glucose solution were added (the first nutrient feed). Second and third feeds, comprising EX-CYTE®, were added at Days 4 and 5 respectively. Temperature, dissolved oxygen, pH, pressure and gas flow rate during the production phase are controlled.

### Example 10

Harvesting the Transformed Cells to Collect the EGFR Antibodies

Cells were removed by cell clarification using depth filtration to obtain a culture broth. The cell broth was then concentrated via tangential flow filtration (TFF) using polyethersulfone membranes. The harvested culture broth was then filtered against a filter having a pore size of 0.2 microns.

### Example 11

Purifying the EGFR Antibodies Produced by the Transformed Cells

The EGFR antibodies of the harvested culture were purified using a sequence of affinity and ion exchange chromatography. In the affinity chromatography step, concentrated cell-free conditioned media is purified over POROS® A50 recombinant Protein A matrix. The concentrated conditioned media was either loaded on an equilibrated Protein A matrix at a pH of 9.00 and washed with equilibration buffer (10 mM sodium phosphate buffer, pH 9.0) to remove unbound impurities or the cell harvest supernatant was loaded on an equilibrated Protein A matrix at a pH of approximately 7.2 and washed with equilibration buffer (10 mM sodium phosphate, 145 mM sodium chloride buffer, pH 7.2). The bound antibodies were eluted from the column of the Protein A matrix using 75 mM acetic acid.

Following elution from the column, a low pH treatment was performed to achieve significant inactivation in virus contamination. Using 1.0 M acetic acid, the pH was lowered to 3.00-3.50 and held for a minimum of 60 minutes. Using 1.0 M Tris base, the pH was then raised to 7.50-8.50.

Further purification was accomplished by concentration and diafiltration against 10 mM sodium phosphate, pH 6.0, via TFF using polyethersulfone membranes. The EGFR antibodies were then processed by anion exchange chromatography over Q Sepharose Fast Flow matrix. The unbound product was eluted using 10 mM sodium phosphate, pH 6.0 buffer. The eluted antibodies were collected as a single fraction. Next, a DV50 virus reduction filtration step was performed to remove a significant amount of virus.

The antibodies were then concentrated and diafiltered against 10 mM sodium phosphate, 145 mM sodium chloride, pH 7.20 via TFF using polyethersulfone membrane.

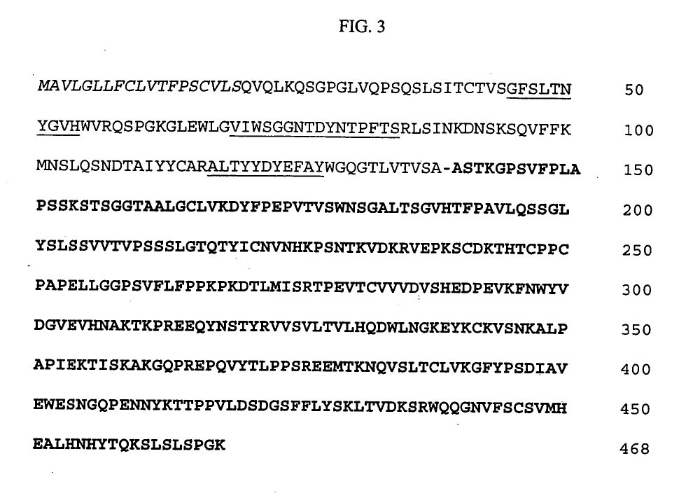

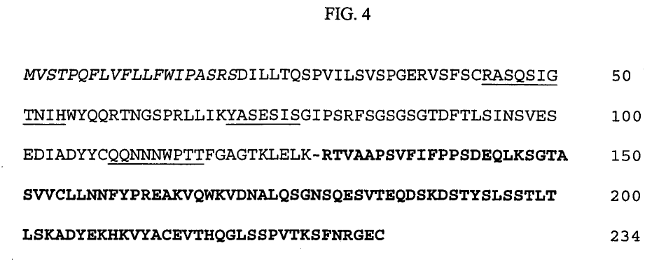

The purified EGFR antibodies were then filtered against a filter having a pore size of 0.2 microns. Set forth is the amino acid sequence of the heavy chain in FIG. 3 and the light chain in FIG. 4. The signal sequences are italicized, the CDRs underlined, and the constant region bolded, with the beginning indicated by (−). The antibodies were then be formulated in phosphate buffered saline with no stabilizers.

The foregoing description and examples have been set forth merely to illustrate the invention and are not intended to be limiting. Since modifications of the disclosed embodiments incorporating the spirit and substance of the invention may occur to persons skilled in the art, the invention should be construed to include everything within the scope of the invention thereof. The disclosures of all citations in the specification are expressly incorporated herein by reference. Furthermore, laboratory protocols applicable to all production methods of the present invention that are described in Sambrook et al., (2000) Molecular Cloning: A Laboratory Manual, 3^{rd} ed., Cold Spring Harbor Laboratory Press, which are incorporated by reference herein.

What is claimed is:

1. A method of producing an antibody specific for epidermal growth factor receptor (EGFR) comprising:
producing transformed cells that express an EGFR antibody;
culturing the transformed cells;
harvesting the transformed cells to collect the EGFR antibody; and
purifying the EGFR antibody.

2. The method of claim 2, wherein culturing the transformed cells comprises:
selecting a transformant having DNA encoding the EGFR antibody;
cultivating the transformant in inoculum cultivation medium to create an inoculum;
scaling-up the inoculum in scale-up medium; and
stirring the inoculum in a production medium to produce and accumulate the EGFR antibody in a culture.

3. The method of claim 2, wherein the inoculum cultivation medium comprises 90% Dulbecco's Modified Eagle's Medium (DMEM) and 10% NCTC-135.

4. The method of claim 2, wherein the inoculum cultivation medium comprises 4 mM glutamine; 7.5 mg/L bovine insulin; 7.5 mg/L bovine transferrin; 1.0 g/L bovine serum albumen (BSA); 30 μM ethanolamine; 40 nM selenium; 30 μM mercaptoethanol; and 150 mg/L oxaloacetate.

5. The method of claim 2, wherein the inoculum cultivation medium comprises 8 mM glutamine; 20.0 mg/L human recombinant insulin; 7.5 mg/L inorganic iron chelator; 1.0 g/L bovine serum albumen (BSA); 30 μM ethanolamine; 40 nM selenium; 30 μM mercaptoethanol; and 150 mg/L oxaloacetate.

6. The method of claim 2, wherein the inoculum cultivation medium comprises 1 μM zinc sulfate and 1 g/L pluronic F68.

7. The method of claim 2, wherein the scale-up medium comprises 14.83 g/L inoculum cultivation medium; 3.55 g/L sodium bicarbonate; 1 g/L pluronic F68; 5 μM methotrexate; 1.25 g/L HySoy, UF; and 5.0 mL/L Excyte VLE.

8. The method of claim 2, wherein the scale-up medium comprises 14.67 g/L inoculum cultivation medium; 3.55 g/L sodium bicarbonate; 2.0 mL/L chelate B; 5 μM methotrexate; 1.25 g/L HySoy, UF; 5.0 mL/L Excyte VLE.

9. The method of claim 2, wherein the production medium comprises 14.83 g/L inoculum cultivation medium; 3.55 g/L sodium bicarbonate; 1.25 g/L HySoy, UF; 5 μM methotrexate; 5.0 mL/L Excyte VLE; and 0.5 μM hydrocortisone.

10. The method of claim 2, wherein the production medium comprises 14.67 g/L inoculum cultivation medium; 3.55 g/L sodium bicarbonate; 2.0 mL/L inorganic iron chelate; 1.25 g/L HySoy, UF; 5 μM methotrexate; 5.0 mL/L Excyte VLE; and 0.5 μM hydrocortisone.

11. The method of any one of claims 1 -10, wherein the EGFR antibody has an amino acid sequence of FIG. 3 and/or FIG. 4.

12. The method of any one of claims 2-10, wherein the DNA encoding the EGFR antibody has a nucleotide sequence of FIG. 1 and/or FIG. 2.


SEQUENCE LISTING (5 sequences)

SEQ ID NO: 1 - DNA, 1407 bp, Homo sapiens
  CDS (1)..(1404)
atggctgtcttggggctgctcttctgcctggtgacattcccaagctgtgtcctatcccaggtgcagctgaagcagtcaggacctggcctagtgcagccctcacagagcctgtccatcacctgcacagtctctggtttctcattaactaactatggtgtacactgggttcgccagtctccaggaaagggtctggagtggctgggagtgatatggagtggtggaaacacagactataatacacctttcacatccagactgagcatcaacaaggacaattccaagagccaagttttctttaaaatgaacagtctgcaatctaatgacacagccatatattactgtgccagagccctcacctactatgattacgagtttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 2 - protein, 468 aa, Homo sapiens
MAVLGLLFCLVTFPSCVLSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 705 bp, Homo sapiens
  CDS (1)..(702)
atggtatccacacctcagttccttgtatttttgcttttctggattccagcctccagaagtgacatcttgctgactcagtctccagtcatcctgtctgtgagtccaggagaaagagtcagtttctcctgcagggccagtcagagtattggcacaaacatacactggtatcagcaaagaacaaatggttctccaaggcttctcataaagtatgcttctgagtctatctctgggatcccttccaggtttagtggcagtggatcagggacagattttactcttagcatcaacagtgtggagtctgaagatattgcagattattactgtcaacaaaataataactggccaaccacgttcggtgctgggaccaagctggagctgaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 4 - protein, 234 aa, Homo sapiens
MVSTPQFLVFLLFWIPASRSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - DNA, 16 bp, Artificial Sequence
  Description of Artificial Sequence Synthetic oligonucleotide
aattctcgagtctaga